(19) United States
(12) Patent Application Publication
(10) Pub. No.: US 2025/0382326 A1
(43) Pub. Date: Dec. 18, 2025

(54) MULTIMODAL ANION EXCHANGE LIGANDS FOR PURIFICATION OF VIRAL PARTICLES AND NUCLEIC ACIDS

(71) Applicant: BOARD OF TRUSTEES OF THE UNIVERSITY ARKANSA, Little Rock, AR (US)

(72) Inventors: Xianghong Qian, Fayetteville, AR (US); Xiaolei Hoa, Fayetteville, AR (US); Sumith Ranil Wickramasinghe, Fayetteville, AR (US)

(73) Assignee: BOARD OF TRUSTEES OF THE UNIVERSITY ARKANSA, Little Rock, AR (US)

(21) Appl. No.: 19/239,402

(22) Filed: Jun. 16, 2025

Related U.S. Application Data
(60) Provisional application No. 63/660,235, filed on Jun. 14, 2024.

(51) Int. Cl. C07K 1/22; C07K 14/005; C12N 7/00

(52) U.S. Cl. CPC C07K 1/22; C07K 14/005; C12N 7/00; C12N 2750/14151

(57) ABSTRACT

A method and surface for separating materials are disclosed. The method includes associating the materials with a surface having a ligand. The ligand includes a hydrophobic unit and an anionic unit. The method further includes forming a material-ligand complex, washing the surface, and eluting the materials from the material-ligand complex. The surface is operational to separate materials. The surface includes a ligand operable to associate with the materials. The ligand includes a hydrophobic unit and an anionic unit.

### CROSS-REFERENCE TO RELATED APPLICATIONS

This patent application claims priority from, and incorporates by reference the entire disclosure of, U.S. Provisional Patent Application 63/660,235 filed on Jun. 14, 2024.

### STATEMENT REGARDING FEDERALLY SPONSORED RESEARCH

This invention was made with government support under 2218054 awarded by the National Science Foundation. The government has certain rights in the invention.

### TECHNICAL FIELD

The present disclosure relates generally to purification of viral particles and nucleic acids, and more particularly, but not by way of limitation, to multimodal anion exchange ligands for purification of viral particles and nucleic acids.

### BACKGROUND

This section provides background information to facilitate a better understanding of the various aspects of the disclosure. The statements in this section of this document are to be read in this light, and not as admissions of prior art.

There is a significant demand for viral vectors for delivery of gene therapies. Adeno-associated virus (AAV) is one of the most effective viral vectors. One of the significant challenges in the commercialization of these virus particle-based therapeutics is inefficient purification operations. The purification of viral vectors such as AAV is further complicated by the presence of full, partially filled, and empty capsids with slightly different hydrophobicity and isoelectric points (pIs). This difference in surface properties is likely due to the effects of encapsulated DNA. The ratio of empty to full capsids can be as high as 10:1. While these empty capsids are naturally occurring contaminants during the virus production process, their presence potentially reduces the effectiveness of the therapy by competing for cell-mediated processes and by serving as impurities that frequently lead to stronger immune responses. Gradient centrifugation is known to provide effective purification of full viral capsids, but it is very difficult, and often impractical, for preparative scale purifications. Separation of full and empty capsids remains a significant challenge.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In a particular embodiment, the present disclosure pertains to a method of separating materials. In some embodiments, the method includes associating the materials with a surface having a ligand. In some embodiments, the ligand includes a hydrophobic unit and an anionic unit. In some embodiments, the method further includes forming a material-ligand complex, washing the surface, and eluting the materials from the material-ligand complex.

In another embodiment, the present disclosure pertains to a surface operational to separate materials. In some embodiments, the surface includes a ligand operable to associate with the materials. In some embodiments, the ligand includes a hydrophobic unit and an anionic unit.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1 illustrates a method of separating materials according to certain embodiments.

FIG. 2 illustrates an example schematic diagram for the development of a multi-model AEX (MMAEX) membrane according to aspects of the disclosure.

FIG. 3 illustrates Fourier transform infrared (FTIR) spectra for a regenerated cellulose (RC) membrane substrate, poly (glycidyl methacrylate) (PGMA) modified, and MMAEX membranes.

FIG. 4 illustrates grafting degree of PGMA on electrospun RC membrane substrate as a function of grafting time from 0 to 240 min.

FIG. 5 illustrates Scanning Electron Microscopy (SEM) images of electrospun RC membranes before (left) and after (right) surface functionalization.

FIG. 6 illustrates Atomic Force Microscopic (AFM) images of electrospun RC membrane (left) and after its functionalization with MMAEX ligand (right) (scan size: 10×10 μm²).

FIGS. 7A-7B illustrate fiber diameter distribution (FIG. 7A) and pore size distribution (FIG. 7B) of electrospun RC membranes before and after surface functionalization based on the SEM images.

FIGS. 8A-8B illustrate the porosity of electrospun RC membranes before and after surface functionalization (FIG. 8A); water contact angles of electrospun RC membrane, after atom transfer radical polymerization (ATRP) polymerization and finally ligand functionalization (FIG. 8B).

FIG. 9 illustrates the zeta potential change as a function of pH in 20 mM bis-tris methan (BTP) buffer for both purified full and empty HEK adeno-associated virus serotype (AAV) 2 capsids.

FIG. 10 illustrates the static binding capacity of bovine serum albumin (BSA) for MMAEX membrane with ATRP time ranging from 20 and 240 min.

FIG. 11 illustrates fast protein liquid chromatography (FPLC) chromatograms during the bind-and-elute runs for MMAEX membranes with two different ATRP grafting times (20 and 240 min) as well as for the unfunctionalized membrane.

FIGS. 12A-12C illustrate chromatographic separation of full and empty AAV2 using gradient elution. Gradient elution peaks of AAV2 and elution buffer conductivity (FIG. 12A), empty and full AAV2 titers in the eluates (FIG. 12B), and full AAV2 capsid purity (FIG. 12C).

FIGS. 13A-13C illustrate 2-step elution of HEK AAV2 using the MMAEX membranes. Samples contain 1.05×10¹² full capsids and 2.26×10¹² empty capsids in 10 mL binding buffer with a loading density of 1.32×10¹⁴ vc/mL.

FIGS. 14A-14B illustrate the fluorescent images of AAV2-infected HEK cells, including cells infected by AAV2 before MMAEX membrane separation (FIG. 14A) and cells infected by AAV2 after MMAEX separation to remove the empty capsids (FIG. 14B).

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

The charge and surface property differences between full and empty capsids have been used to remove empty capsids and enrich the full capsids using membrane-adsorbers such as anion-exchange chromatography (AEX) or multimodal AEX (MMAEX) chromatography. The separation is difficult with typically less than 50% yield for commercial columns or membranes. CIMac™ AAV, a monolith AEX has improved full/empty separation, but it is very costly and not easily scalable. By carefully tuning the ligand properties and elution conditions on a membrane substrate or on resin beads, it is possible to separate full capsids from empty ones using a novel in-house fabricated MMAEX ligand.

This present disclosure describes the development of a novel chromatographic multimodal anion exchange (MMAEX) ligand for the separation of empty and full adeno-associated virus (AAV) capsids or other viral particle capsids. The ligands are grafted directly on membrane or bead substrates, which can be commercially available regenerated cellulose (RC) membranes, electrospun nanofiber RC membranes, or other appropriate membrane substrates, as well as resin beads, or other chromatographic stationary phases.

Additionally, the present disclosure describes the development of a unique MMAEX membranes using nanofiber RC membrane substrate and the grafting of the MMAEX ligand directly on the surfaces of the membrane substrate. It should be noted that other stationary substrates are readily envisioned.

Various embodiments of the present disclosure illustrate the fabrication of this unique MMAEX ligand grafted directly on the membrane substrates. In brief, the first step during ligand development is the immobilization of an initiator for the subsequent polymerization reaction. The second step includes the grafting of poly (glycidyl methacrylate) (PGMA) polymer chains via controllable atom transfer radical polymerization (ATRP) methods. Phenylimidazole is then covalently bonded to the epoxy groups on the PGMA chains. The final steps involve the formation of quaternary amines by Menshutkin reaction. The final development of phenylimidazole cations as a multimodal anion exchange ligand is new and unprecedented.

Optimizing ligand chain length and ligand chain density provides for the successful development of this MMAEX membrane. The MMAEX ligands described herein, have both the hydrophobic (phenyl) and anion exchanger (quaternary amine) groups, which work synergistically to selectively bind and elute the full and empty capsids which have minor differences in their charge and hydrophobicity during appropriate chromatographic conditions. This type of MMAEX ligands has both aromatic hydrophobic groups conjugated with the imidazole positively charged groups, providing a better separation resolution for the full and empty viral capsids which have small differences in hydrophobilicty and charge. The ligands disclosed herein that contain conjugated aromatic groups and imidazole groups are unique and can be further tuned for specific separation needs. Ligand property tunable by increasing/changing the aromatic conjugations (more or few aromatic rings) or aliphatic groups (long or short R- groups) on the imidazole ring.

The membranes with unique MMAEX ligands have high protein binding capacity and recovery rate. Bovine serum albumin (BSA) was chosen as a modal protein to test the static and dynamic protein binding capacities (BSA has similar isoelectric point to AAV particles). Membranes were incubated with BSA solution for 7 hours. The BSA concentrations before and after static binding were measured and used for the calculation of the static binding capacity. The longer grafting time led to longer PGMA chains and therefore higher density of MMAEX ligands which leads to a higher static BSA binding capacity of over 100 mg/mL BSA up to 240 min of grafting time.

The capability of separating empty and full AAV capsids of the MMAEX membranes has been verified by bind-and-elute of a 1:1 mixture of empty and full AAV capsids. The loaded particle numbers of empty and full AAV capsids were 2×10¹¹. Following the wash step, a two-step elution was applied, where one elution peak can be seen for each step elution. The first elution peak contains largely empty AAV capsids, with a A260/280 ratio at around 0.68. The second elution peak contains full capsids with a A260/280 ratio at around 1.26. Furthermore, the purity of full AAV capsids was determined by ELISA and qPCR assays. The results show that the purity of full AAV in the second elution peak reached 88% from original 50% in the feed. The purity could be further improved by optimizing the elution conditions. Compared to conventional AEX or exiting MMAEX for AAV full and empty capsid separation, the present disclosure illustrates a unique design of the MMAEX ligand which has superior performance for the separation. Table 1 illustrates the dynamic binding capacity and recovery of BSA.

TABLE 1
The dynamic binding capacity and recovery of BSA.

| | Bonded | Eluted | Membrane | Dynamic Binding | Recovery |
|---|---|---|---|---|---|
| Grafting | BSA | BSA | Volume | Capacity | Rate |
| Time | (mg) | (mg) | (ml) | (mg/ml) | (%) |
| 20 min | 0.84 | 0.81 | 0.012 | 67.59 | 96.32 |
| 240 min | 1.29 | 1.31 | 0.016 | 77.51 | 101.88 |

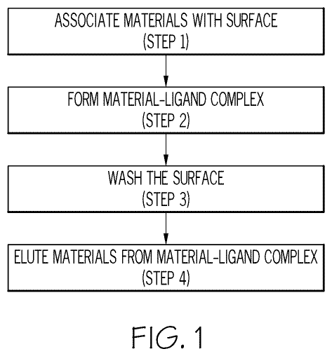

In view of the aforementioned, the present disclosure relates to methods and surfaces for separating materials. As illustrated in FIG. 1, in some embodiments, the present disclosure pertains to a method of separating materials. In some embodiments, the method may include associating the materials with a surface having a ligand (Step 1). In some embodiments, the ligand includes a hydrophobic unit and an anionic unit. In some embodiments, the method may include, without limitation, forming a material-ligand complex (Step 2), washing the surface (Step 3), and eluting the materials from the material-ligand complex (Step 4).

In an additional embodiment, the present disclosure pertains to a surface operational to separate materials. In some embodiments, the surface includes a ligand operable to associate with the materials. In some embodiments, the ligand includes a hydrophobic unit and an anionic unit.

The methods and surfaces of the present disclosure can have numerous embodiments. For instance, the present disclosure may utilize various methods to associate materials with surfaces. For example, in some embodiments, the associating may include incubating the materials with the surface. In certain embodiments, the associating may include flowing the materials through the surface.

Additionally, the methods and surfaces of the present disclosure may be utilized to separate various materials. For example, the materials may include, without limitation, nucleic acids, DNA, RNA, mRNA, miRNA, monoclonal antibodies, vaccines, therapeutic proteins, viruses, viral particle capsids, or combinations thereof. In certain embodiments, the materials may include viral particle capsids. In some embodiments, the viral particle capsids may include, without limitation, full capsids, empty capsids, adeno-associated virus (AAV) capsids of various serotypes, or combinations thereof. In certain embodiments, the AAV capsids of various serotypes may include, for example, AAV2 capsids, AAV5 capsids, AAV9 capsids, or combinations thereof.

In addition, the methods of the present disclosure may utilize various surfaces to separate materials. Furthermore, the surfaces of the present disclosure may be in various forms. For example, in some embodiments, the surface may include, without limitation, beads, resin beads, columns, membranes, regenerated cellulose (RC) membranes, electrospun nanofiber RC membranes, chromatographic stationary phases, monolith surfaces, organic substrates, inorganic substrates, or combinations thereof.

The surfaces of the present disclosure may include various ligands for separation of the materials. For example, in some embodiments, the ligand includes a hydrophobic unit and an anionic unit.

In some embodiments, the hydrophobic unit of a ligand may include, without limitation, aromatic groups, phenyl groups, aromatic conjugated groups, aliphatic groups, or combinations thereof.

In some embodiments, the anionic unit of a ligand may include quaternary amine groups. In certain embodiments, the anionic unit includes imidazole groups.

In certain embodiments, the ligand has a cationic charge. In some embodiments, the ligand includes phenylimidazole cations. In some embodiments, the phenylimidazole cations include 2-methyl-phenylimidazole, ethyl-phenylimidazole, phenylimidazoles with an alkane (e.g., C3- or higher) side chain, aromatic conjugated imidazole groups, or combinations thereof.

The methods and surfaces of the present disclosure have various advantages. For example, the two ligand-substrate interaction mechanisms work synergistically, providing high separation resolution. Additionally, the ligand properties are tunable, which allow for the tailoring for specific separation needs. Furthermore, the methods and surfaces of the present disclosure provide high recovery rate, flexibility in substrate selection, and the potential for a boarder range of applications.

Moreover, the methods and surfaces of the present disclosure have a novel conjugation of aromatic hydrophobic groups and imidazole charged groups that enables better separation of empty and full AAV capsids than conventional AEX and other MMAEX membranes/resins. Additionally, a higher binding capacity and shorter operation time can reduce operation cost.

Moreover, the type of MMAEX ligands is tunable and flexible in substrate selection. As such, various products can be developed. For instance, in some embodiments, the ligands of the present disclosure may be grafted to various substrates including, but not limited to, membrane, monolith and resin. These developed products have broader applications in addition to separating empty and full AAV capsids. It is envisioned that the production of other biopharmaceuticals can benefit from the methods and surfaces of the present disclosure including, for example, vaccines, monoclonal antibodies, nucleic acids and other viral vectors.

### ADDITIONAL EMBODIMENTS

Reference will now be made to more specific embodiments of the present disclosure and experimental results that provide support for such embodiments. However, the disclosure below is for illustrative purposes only and is not intended to limit the scope of the claimed subject matter in any way.

### Fabrication of Multi-Modal Anion Exchange Membrane for Chromatographic Purification of Full Adeno-Associated Viral Capsids

A multi-modal anion exchange ligand 2-methyl-phenylimidazole has been successfully functionalized on highly porous electrospun regenerated cellulose membrane substrate for the separation of full and empty adeno-associated virus (AAV) capsids. The functionalized membrane has an average pore size of 0.5 μm and exhibits both charge and hydrophobic functionality. The ligand is able to distinguish the subtle physio-chemical differences between the full and empty viral capsids. The multi-modal anion exchange membrane is able to separate the full capsids from affinity captured mammalian AAV2 using both gradient and two-step elution methods with superior performance. A full capsid recovery of 72.9% at 85.3% purity with gradient elution, or 70.3% recovery at 92.2% purity with two-step elution can be achieved at significantly higher viral capsid loading of ˜0.5-1.3×10¹⁴ particles per milliliter of membrane volume compared to previous studies. Moreover, AAV2 capsids demonstrate similar transduction efficiency before and after the purification with the functionalized membrane.

Introduction. Recombinant adeno-associated virus (rAAV) has emerged as one of the best delivery vectors for gene therapy products due to its relative low pathogenicity and long-term effects. Gene and cell therapy have seen unprecedent growth during the past decade for treating a variety of human diseases. By the end of 2023, a total of 7 AAV-based gene therapy products had been approved by Food and Drug Administration (FDA). By the end of 2024, the approved gene therapy products have increases to 19. Currently the total number of approved gene and cell therapy products reached more than 40.

AAV is a single-stranded DNA virus. However, DNA is not always properly incorporated into the capsids during the virus particle assembly. Consequently, host cells produce empty and full AAV capsids and the ratio of empty to full AAV capsids can be as high as 10:1. While these empty capsids are naturally occurring contaminants during the virus production process, their presence potentially reduces the effectiveness of the therapy by competing for cell-mediated processes which can also lead to the undesired immuno-responses. To increase the treatment efficacy, administration of high doses of rAAV (˜10¹³-10¹⁴ capsids) has been used as a strategy to achieve improved transduction. However, hepatotoxicity or liver toxicity has been observed due to the high number of viral particles pass through the liver upon entering circulation. Therefore, it is important to remove empty capsids from the final product.

Full and empty capsids have almost the same 18-24 nm size range making size-based separation almost impossible. However, the full capsids have a slightly higher density at 1.40 g/cm³ than the density at 1.32 g/cm³ of the empty capsids. This difference in density has been used for their separation with ultracentrifugation. However, ultracentrifugation is rather energy intensive and difficult to scale up. Besides density, there are additional physio-chemical differences between the full and empty capsids. For example, the full capsids are slightly more negatively charged and more hydrophilic than the empty capsids due to the DNA inside the full capsids. The difference in isoelectric points (pI) between the full and empty capsids is about 0.5 pH unit or less. This difference in charge has been used for the separation of the full and empty capsids using anion exchange (AEX) chromatographic processes at the chromatographic polishing step. Since the difference in charge is small, the separation of full and empty capsids with AEX remains challenging, typically requiring carefully selected buffer conditions using a bind-and-elute operation. At binding or low conductivity buffer condition, the more negatively charged full capsids have a stronger binding interaction with the positively charged amine ligands than the corresponding empty ones. During elution, empty capsids are eluted first followed by the full capsids at higher conductivity buffer condition. Different elution strategies have been investigated, including isocratic, gradient and two-step elution approaches for full and empty capsid separation.

Commercial AEX membrane has demonstrated ˜70% full capsid recovery at ˜70% purity under optimized conditions using design of experiments (DOE). This study used AAV loading density of ˜10¹³ capsids per milliliter (mL) of membrane volume. This recent development of a novel multi-model AEX (MMAEX) membrane shows that ˜90% full capsid recovery at ˜90% purity can be achieved with gradient elution. Moreover, with a two-step elution, 88% full capsid recovery at 75% purity can also be achieved. However, only purified full and empty AAV2 stock from insect cell line Sf9 was used in this early study. The loading density is also slightly low at ˜10¹² capsids/mL for gradient elution but is similar to previous study at ˜10¹³ capsids/mL for the two-step elution. Earlier studies with a CIMac™ AEX monolith, 70% full capsid recovery at 90% purity has also been obtained using gradient elution. However, the loading density is only 5×10¹¹ capsids/mL, much lower than a previous study with MMAEX membrane. Moreover, AEX process with monolith is more expensive and not as easily scalable compared to membrane process.

Here the capability of this novel MMAEX membrane is further demonstrated by separating the full and empty capsids with Applicant's in-house produced AAV2 feedstock from HEK293. A ˜70% full capsid recovery at 92% purity has been achieved with a significantly higher loading density of 1.3×10¹⁴ capsids/mL, over 200 times more than the previous CIMac™ AEX monolith study. Moreover, infectivity experiments using pre- and post-MMAEX membrane chromatographic separated AAV2 exhibit similar multiplicity of infection (MOI) indicating the suitability of Applicant's membrane for AAV capsid separations.

Materials and Reagents. The following chemicals and solvents were purchased from Sigma-Aldrich (St Louis, MO): cellulose acetate (39.8 wt % acetyl, average molecular weight 30,000), N,N-dimethyl acetamide (DMAC, 99%), triethylamine (TEA, 99%), 2-bromoisobutyryl bromide (2-BIB, 98%), methanol (99.9%), N,N,N′,N″,N″-pentamethyldiethylenetriamine (PMDETA, 99%), glycidyl methacrylate (98%), copper (I) chloride (98%), 2-phenylimidazole (99%), sodium phosphate dibasic (ACS reagent, 99%), sodium phosphate monobasic (ACS reagent, 99%), glycerin (electrophoresis grade, >99%), 0.2 μm PES filters, diafiltration centrifuge tubes Pierce™ protein concentrators (PES, 10K MWCO, 6.0 mL), POROS™ GoPure™ AAVX affinity column, acrylamide (synthesis grade, >99%), glycerol (molecular biology grade, ≥99.0%), glycerin (electrophoresis grade, >99%), formalin (37%).

The following chemicals and solvents were purchased from VWR (Randor, PA): acetone (99.5%), bovine serum albumin (98%, molar weight ˜66 kDa), bis-tris propane (≥99.9%), magnesium chloride (>99.9%), sodium chloride (ACS reagent, 99%), tetrahydrofuran (anhydrous, ≥99.9%), triethylamine (>99%), iodomethane (>99%), 2,6-lutidine (>98%), tris-base (>99%), sodium chloride (ACS reagent, 99%), and PBS tablet (biotechnology grade), magnesium chloride (>98%), sodium dodecyl sulfate (SDS) (proteomics grade, >99.9%), ammonium persulfate (APS) (ACS, >98%), tris-HCl (bioreagent, >99.0), N,N,N′,N′-tetramethyl ethylenediamine (TEMED) (99%).

The following chemicals and reagents were purchased from Thermo Fisher Scientific (Waltham, MA, USA): Gibco® viral vector HEK medium, GlutaMAX™ supplement, AAV-MAX transfection kit, AAV-MAX control plasmids kit, and AAV-MAX lysis buffer, pluronic F68, NuPAGE™ LDS sample buffer (4×). DI water was produced by Thermo Scientific Barnstead Smart2Pure UV/UF System.

DNase and DNase buffer (10×) were purchased from Promega (Madison, WI, USA), nuclease-free deionized (DI) water was purchased from OMEGA BIO-TEK (Norcross, GA, USA), iTaq™ universal SYBR green supermix and PCR tubes/caps were purchased from Bio-Rad (Hercules, CA, USA), and forward and reserve primers were purchased from Integrated DNA Technologies (Coralville, IA, USA). The AAV2 genome titers were quantified by a Bio-Rad CFX Connect™ Real-Time System (Hercules, CA, USA). The AAV2 ELISA assay kit was purchased from Progen Biotechnik (Heidelberg, Germany). The microplate reader was purchased from SpectraMax (San Jose, CA, USA). Asahi Kasci (Tokyo, Japan) provided BioOptimal™ MF-SL for AAV lysate clarification.

Methods: Fabrication of Electrospun Regenerated Cellulose (RC) Membrane. The fabrication of electrospun RC base membrane has been described previously. Briefly, the solvent for the dope solution consists of acetone and N, N-dimethyl acetamide (DMAC) (2:1, w/w). The dope solution was prepared by cellulose acetate to prepared solvent at a concentration of 14.2 wt % with stirring until the cellulose acetate was fully dissolved. A syringe with a 21-gauge needle were used for electrospinning at a flow rate of 0.3 mL/h with a syringe pump. The distance between the needle tip and the aluminum foil collector was 15 cm with applied DC voltage of 13.5 kV. The humidity was controlled at 75±5%. Electrospinning was conducted for 9 hours. The nanofiber membrane collected on the aluminum foil was placed in a vacuum oven and dried at 40° C. Finally, membrane discs, 47 mm each were cut and placed in 200 mL 0.1 M NaOH solution overnight to hydrolyze the acetate groups generating the RC nanofibrous membrane. The membranes were rinsed in DI water and then dried in a vacuum oven at 40° C.

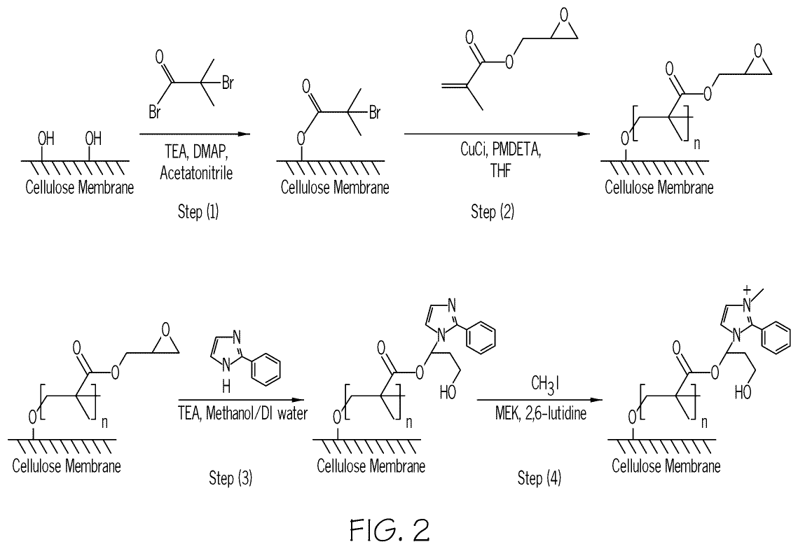

Grafting of Multi-Modal AEX Ligands on RC Membrane Substrate. FIG. 2 shows the schematics of membrane with functionalization a multi-modal AEX (MMAEX) ligand for AAV full and empty capsid separation using surface-initiated atom transfer radical polymerization (SI-ATRP). The surface modification process consists of initiator immobilization, grafting of poly (glycidyl methacrylate) (PGMA), conjugation of multi-modal ligand 2-phenylimidazole to PGMA, and N-methylation of the ligand by the addition of CH₃I to 2-pheylimidazole to form the positively charged 1-methyl-phenylimidazole ligand.

Initiator Immobilization. The initiator immobilization solution was prepared by mixing 0.35 mL of triethylamine (TEA) and 0.25 mL of 2-bromo-2-methylpropionyl bromide (2-BiB) in 10 mL acetonitrile. In addition, 5.25 mg of 4-mimethylaminopyridine (DMAP) were also dissolved in the solution. One piece of cellulose nanofibrous membrane was then immersed into the solution for 3 h. The reaction was terminated by removing the membrane from the reaction solution and rinsing it with acetonitrile, ethanol and DI water.

Grafting of Poly (Glycidyl Methacrylate) (PGMA) via ATRP. A total of 12 mL of glycidyl methacrylate (GMA) and 13 mL of tetrahydrofuran (THF) were mixed in a three-neck flask. A total of 47 μL of pentamethyldiethylenetriamine (PMDETA) and 22.3 mg of copper (I) chloride (CuCl) were added into the solution mixture after purging by Ar for 20 min, followed by sonication for 30 min. One initiator immobilized membrane was added into a separate three-neck flask and deoxygenated by argon flushing. The prepared solution mixture was then added to the flask containing the membrane. The ATRP reaction started for a predetermined length of time. The reaction was terminated by removing the membrane from the vessel. The membrane was then rinsed with methanol and DI water.

Conjugation of 2-Phenylimidazole to PGMA. A total of 360.4 mg of 2-phenylimidazole and 348 μL of TEA were added into the 20 mL of methanol/water (50:50, v/v) mixture. One piece of PGMA-modified membrane was then immersed in the reaction solution. The reaction was carried out at 60° C. It was terminated by removing the membrane from the reaction solution and rinsing it with methanol and DI water 3 times each.

N-Methylation to Form 1-Methyl-Phenylimidazole Multi-Modal Ligand. A total of 145 μL of 2,6-lutidine and 78 μL of iodomethane were added into 10 mL of 2-butanone. One piece of phenylimidazole-PGMA-modified membrane was then immersed in the reaction solution. The reaction was carried out at 60° C. and terminated by removing the membrane from the reaction solution and rinsing it with methanol and DI water. Thereafter the MMAEX membrane is ready for use.

GraftingDegree Determination. Grafting degree of PGMA-modified membranes was calculated according to Equation (Eq.) 1:

$$Grafting degree = \frac{{m}_{1} - {m}_{0}}{{m}_{0}} \times 100 \% \qquad ( Eq . 1 )$$

where m₀ is the mass of the membrane before grafting and m₁ is the mass of the membrane after grafting PGMA.

Fourier Transform Infrared (FTIR) Spectroscopy. FTIR spectroscopy was conducted with a Shimadzu IRAffinity-1S spectrometer equipped with an attenuated total reflectance (ATR) accessory (Shimadzu, Japan). The FTIR spectra were recorded at the resolution of 4 cm⁻¹, with the scan range from 600 to 4000 cm⁻¹. The data were analyzed using the software, IRsolution (Shimadzu, Japan). Before performing the FTIR run, membranes were dried at 60° C. to remove any water.

Scanning Electron Microscopy (SEM). The membrane samples were imaged by a scanning electron microscope (SEM) (FEI Nova Nanolab 200 Duo-Beam Workstation, Hilsboro, Oregon, USA). SEM images were taken at a magnification of 10000. The nanofiber diameter and pore size were measured using ImageJ (version 1.54 g, Oct. 18, 2023).

Porosity Determination. The porosity of the membrane samples was determined using the wet method. The dry weight of one coupon of membrane was recorded first. The membrane was wetted by immersing it in DI water. It was then taken out and the excessive water on both sides of the membrane was removed by a Kimwipe tissue. The weight of the wet membrane was measured. In addition, the thicknesses of the membrane at four endpoints were measured and the average was used to calculate the membrane volume. The porosity was calculated by Eq. 2:

$$Porosity = \frac{{m}_{w} - {m}_{d}}{ρπ {r}^{2} t} \qquad ( Eq . 2 )$$

where m_{w} is the wet membrane weight, ma is the dry membrane weight, p is the DI water density (1.0 g/mL), r is the membrane radius (23.5 mm), and t is the average thickness of the membrane.

Atomic Force Microscopic (AFM) Imaging of Membranes. The surface morphologies of the fabricated electrospun membrane before and after ligand surface modification were measured using an Atomic Force Microscope (Bruker, Billerica, MA, USA). The samples were measured in peak force tapping mode (ScanAsyst mode) with a silicon nitride ScanAsyst-Air probe. The scan rate was set at 0.4 Hz, and the scan size was 10×10 μm.

Contact Angle Measurement. Static contact angle measurements between membrane surface and water drop were conducted by the sessile drop method. The set up includes an optical angle meter (OCA 20, Future Digital Scientific Corp., NY) and a dosing needle. Membranes were cut into small pieces and fixed on a flat glass chip with double side tape for measurements. The dosing volume is 5 μL. Each result was reported by averaging at least 3 measurements at random locations and the standard deviations were shown as error bars.

Zeta Potential Measurement. The zeta potential of purified AAV2 capsids was measured using Anton Paar Litesizer 500 (Graz, Austria). The purity of the separated empty HEK AAV2 was ˜90%, and the full HEK AAV2 was ˜95% in bis-tris-propane (BTP) buffer at pH 9. A 10 kD diafiltration centrifuge tube was used to replace the original buffer with 20 mM BTP buffer (99.9% removal of the original buffer) reaching a final viral capsid concentration of ˜10¹⁰ capsids/mL. The pH of the BTP buffer was varied by titration using 6 M HCl or 2 M NaOH. After the cuvette was filled with the AAV sample, the zeta potential measurement was carried out by the Kalliope software (version 1.2.0, Anton Paar, Austria) in the pH range between 3 and 9.

AAV Production and Clarification. HEK 293 suspension cells were cultivated using a complete growth medium Gibco® viral vector HEK medium containing 2% (v/v) GlutaMAX™ supplement. The medium was pre-warmed to 37° C. before inoculation. The initial cell seeding density was set at 0.3×10⁶ cells/mL. The culture was maintained in a CO₂ shaking incubator at 37° C. and 82% relative humidity under continuous shaking at 250 rpm. When the cell density reached 5.0-6.0×10⁶ cells/mL, the culture was diluted with fresh medium to adjust the final cell density to 3.0×10⁶ cells/mL for AAV2 capsid production. AAV2 plasmids including pAAV2 gene, pAAV-rep2/cap2, pHelper at 1.0:2.0:0.5 ratio were transfected into the HEK 293 cells for AAV2 vector production. The culture was incubated for 72 hours before harvesting. During harvesting, a 10% (v/v) AAV-MAX lysis buffer was added to the cell medium and incubated for 4 hours to lyse the cells and release AAV2 capsids into the medium. Tangential microfiltration using BioOptimal™ MF-SL microfilter was performed to remove the cells and cell debris from the AAV2 lysate. Thereafter affinity capture was performed with the clarified feed to further purify the AAV2 vectors.

AAV Affinity Capture and Buffer Exchange. POROS™ GoPure™ AAVX affinity column was used to capture AAV capsids from the clarified AAV2 feed solutions. Before affinity capture, the clarified AAV feed was first filtered by a 0.2 μm filter. The binding buffer was 20 mM Tris containing 150 mM NaCl and 0.01% pluronic at pH 8.0, while the elution buffer was 100 mM glycerin containing 150 mM NaCl and 0.01% pluronic at pH 2.5. The flow rate throughout the fast protein liquid chromatography (FPLC) operation was kept at 1.0 mL/min. During the process, the affinity column was first washed with DI water for 20 mins, followed by equilibration with the binding buffer for another 20 mins. The filtered AAV sample was then pumped through the column, followed by the wash step with the binding buffer for 20 min to remove unbound materials. During the elution step, the eluate was collected every 1 mL fraction into tubes pre-filled with 0.15 mL of 1.0 M Tris at pH 8.4.

The elution buffer contained glycine, which was shown to affect the binding of AAV capsids during the subsequent chromatographic full/empty capsid separation. As a result, diafiltration was performed using a 10 kD molecular cutoff (MWCO) diafiltration filter to remove glycine from the feed samples. AAV capsids were buffer exchanged into PBS containing 0.01% pluronic. A 3 mL aliquot of the AAV sample was added into the diafiltration filter tube and then centrifuged at 3000 g until the volume was reduced to 0.3 mL. Afterward, 2.7 mL of diafiltration buffer was added and the sample was centrifuged again. A total of 5 diafiltration cycles were repeated, replacing 99.999% of the elution buffer with the PBS buffer. The resulting AAV sample was aliquoted into 200 μL tubes and stored at −80° C. for future use.

Chromatographic Separation of Full and Empty AAV2 Capsids. All experiments were conducted using an ÄKTA Pure chromatography FPLC system (Cytiva, Marlborough, MA), and the data were collected and analyzed by the software Unicorn 7.3. A stack of 4 membrane discs with total volume of 0.025 mL was placed in a CIM® module (BIA Separations, Inc., Wilmington, DE) and equilibrated with binding buffer for 30 min at a flow rate of 0.5 or 0.25 mL/min. The binding buffer was 20 mM bis-tris propane (BTP) with 2.0 mM MgCl₂ at pH 9.0. The elution buffer was 20 mM BTP with 2.0 mM MgCl₂ with varying concentration of NaCl at pH 9.0 depending on the elution condition.

Gradient Elution for Full/Empty AAV2 Capsid Separation. The AAV feed sample was prepared by pipetting predetermined amount of affinity purified and buffer exchanged HEK AAV2 capsids to the BTP binding buffer (pH 9, conductivity 1.5 mS/cm). A total of 10 mL of the AAV feed sample was loaded onto the MMAEX membrane stack followed by 5 mL of washing with the binding buffer. Thereafter a total of 10 mL of elution buffer was bumped through the membrane module via gradient elution with conductivity increased gradually from 1.5 mS/cm to 15 mS/cm at the end of the elution step. The eluates were collected in 0.5 mL fractions. The full AAV2 capsid titers in the eluates were determined by qPCR, and the total AAV2 capsid titers were quantified by ELISA.

Two-Step Elution for the Separation of Full and Empty AAV2 Capsids. The same procedure was used to prepare for the feed samples for the two-step elution for the full and empty capsid separation. The binding buffer used was the same as that from the gradient elution. For two-step elution process, the elution buffers contained different amounts of NaCl by mixing different ratios of binding buffer with the elution buffer (BTP buffer 1000 mM NaCl, pH 9, conductivity 82.9 mS/cm) to elute the bound AAV capsids using two-channel pump with a mixer. The buffer conductivity was controlled by controlling the percentage of elution buffer in the mixture. Three different two-step elution runs were performed with first-step elution buffer conductivity at 8.0, 8.5 and 9.0 mS/cm respectively. The second-step elution buffer conductivity was kept at 14 mS/cm for all three runs.

AAV Capsid Titer Quantification. The full capsid titers of the samples are determined by qPCR assay as described in more detail in previous work. The total capsid titers of the samples are quantified using ELISA assay was also described previously. The empty capsid titers are then calculated by deducting full capsid titer by the total capsid titer. UV absorption at 260 and 280 nm ratio A260/A280 is often used as a qualitative measure of the full capsid content in the samples. If A260/A280 is close to 0.7, then the sample is probably protein in nature indicating empty capsid presence. The higher the A260/A280 ratio becomes, the more nucleic acid composition the sample has. For example, for pure DNA, UV absorption ratio reaches 1.9-2.0. For full capsid containing both protein and nucleic acid, the A260/A280 ratio could be in the range of 1.2-1.3. For mixed full and empty capsid samples, the A260/A280 ratio can be between 0.7 and 1.3 depending on the percentage of the full capsids in the sample.

AAV Infectivity Assay. HEK cell culture was prepared with seeding density of 0.04×10⁶ cells/mL in growth medium Gibco® viral vector HEK medium with 2% (v/v) GlutaMAX™ supplement. A total of 5 mL of the cell culture was then added to a 50 mL sterile centrifuge tube (2.0×10⁵ HEK cells). The full capsid titer was quantified by qPCR before the infectivity assay. The AAV2 sample was first filtered using a 0.2 μm sterile filter. Then about ˜1.0×10¹⁰ full AAV2 capsids were added to the 5 mL cell culture. The volume of the added AAV2 sample depends on its AAV2 titer, but the AAV2 sample volume should be less than 5% of the cell medium, which is equivalent to 250 μL. The cell culture transfected with AAV viral particles was incubated in a CO₂ shaking incubator without the cap at 250 rpm. The incubation temperature and relative humidity were kept at 37° C. and 82% respectively.

After 48 hours of incubation, the tube was centrifuged at 1000 rpm for 5 min, the cell medium was carefully pipetted out, and 1 mL of PBS was added to resuspend the cell pellet. The cell density of the 1 mL suspension was measured by microscopic direct cell counting using a counting chamber. The HEK cells were imaged using a Nikon Eclipse Ts2 fluorescence microscope (Melville, NY). Both the fluorescence and bright-field channels were imaged. The fluorescent HEK cells and total HEK cells in the same scope were counted. Thus, the infection percentage was calculated by Eq. 3:

$$Infection percentage = \frac{Number of infected HEK cells}{Number of total HEK cells} \times 100 \% \qquad ( Eq . 3 )$$

The multiplicity of infection (MOI) was calculated according to Eq. 4:

$$M O I = \frac{Full AAV capsid number}{Number of infected HEK cells} \qquad ( Eq . 4 )$$

where full AAV capsid number was quantified by qPCR.

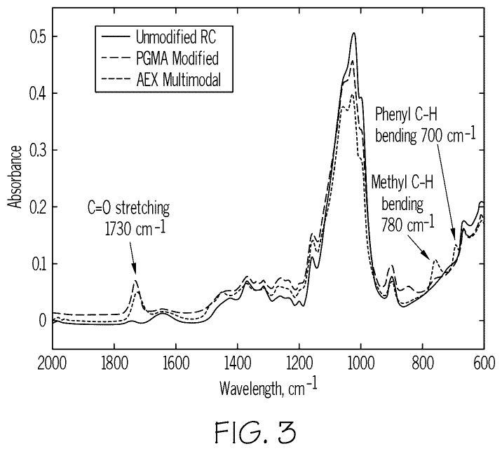

Results and Discussion: Fourier Transform Infrared (FTIR) Spectroscopy. FIG. 3 shows the FTIR spectra of the electrospun RC membrane substrate after ATRP modification with PGMA and the last step functionalization to form the MMAEX ligands. It can be seen that both PGMA modified and MMAEX membranes have a strong peak at 1730 cm⁻¹ due to carbonyl C═O stretching which is absent in the base membrane. This indicates ATRP grafting of PGMA is successful. The final functionalized MMAEX membrane has additional weak absorption at 700-780 cm⁻¹ indicating the presence of phenyl and methyl groups and successful conjugation of 2-phenylimidazole to PGMA.

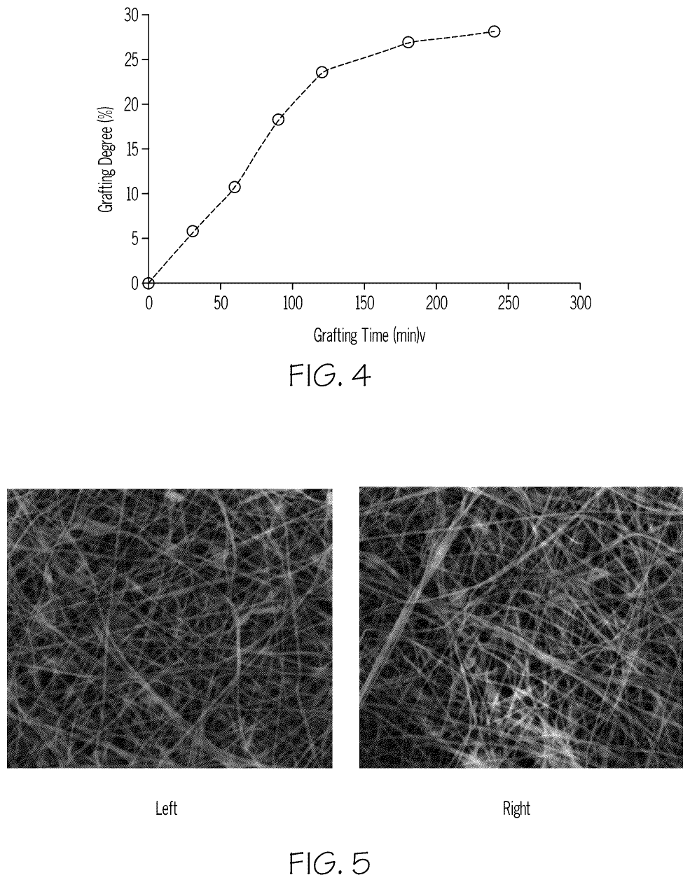

GraftingDegree. FIG. 4 shows the grafting degree of PGMA as a function of ATRP reaction time. It can be seen that grafting degree increases almost linearly from 0 to 23% with polymerization time from 0 to 120 min. However, polymerization slowed down with further increase in grafting time to 240 min. It is possible that the radicals at the chain ends could be buried inside the polymer for the longer chains limiting chain growth. In addition, radicals at the long chain ends could combine and therefore terminate the polymerization process. For the subsequent membrane characterization and chromatographic studies except for the dynamic binding studies of BAS, membranes with 30 min ATRP reaction at ˜5% grafting degree were used in order to minimize the effects of surface functionalization on membrane structures.

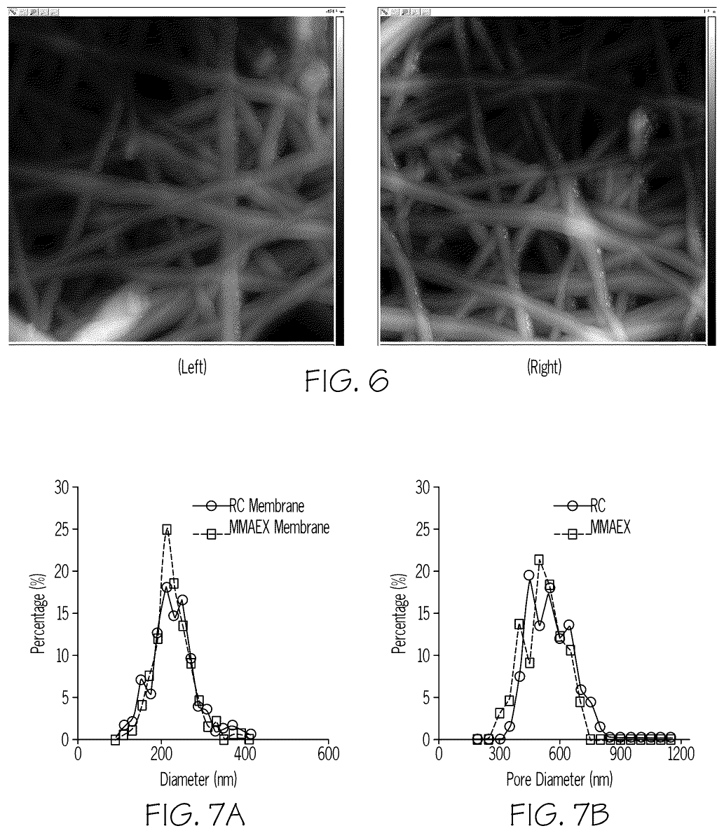

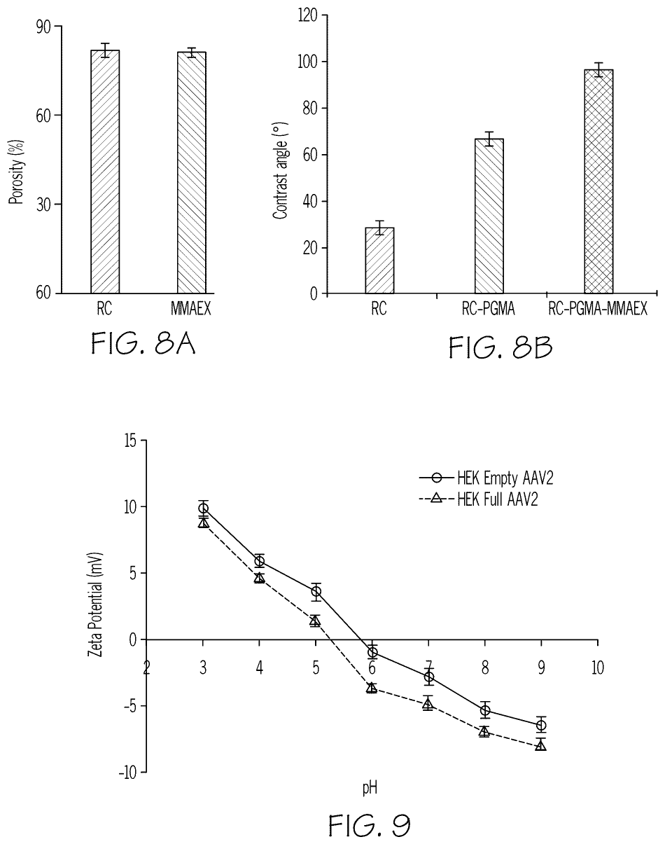

Surface Characterization of Nanofibrous Membranes. SEM images were taken for the electrospun RC membrane before and after 30 min of ATRP for grafting PGMA and subsequent functionalization with MMAEX ligand as shown in FIG. 5. It can be seen that nanofibers are randomly but uniformly arranged. After surface modification, surface morphology remains almost the same. The higher resolution AFM images shown in FIG. 6 also suggest that surface topology did not change before and after surface functionalization.

Besides nanofiber arrangement and surface morphology, nanofiber diameters were estimated based on the SEM images. A total of 200 distinct spots were evenly selected across the surface. The diameters were categorized into different ranges between 100-400 nm with 20 nm increment. The percentages of diameters in their respective intervals were then calculated and plotted. Fiber diameter distributions are measured both before and after surface modification as shown in FIG. 7A. It can be seen that the fiber diameters lie between 100 and 400 nm range for membranes both before and after functionalization with the peak at around 210 nm. After surface modification, the peak was observed to be slightly broadened. This shows that surface modification only had minimal impact on membrane structure and morphology. For pore size measurement based on SEM images, 100 pores were evenly selected across the surface, the pore size values were categorized into different ranges between 200-1000 nm at 50 nm increment. The numbers of pore sizes at their respective ranges were determined. FIG. 7B shows membrane pore size distribution. The pore size ranges from 300 to 800 nm with the peak at 500 nm for the unmodified electrospun RC membranes. After surface functionalization with MMAEX ligand, the pore size distribution shifted slightly to the lower end with the pore size ranging from 250 to 750 nm. However, the peak positions for the pore size distributions remain more or less the same.

Besides fiber diameter and pore size distributions of the fabricated electrospun RC membranes before and after surface functionalization, membrane porosity was also measured. Porosity measurement is based on the wet method as described earlier. As shown in FIG. 8A, the electrospun membranes are highly porous with porosity estimated to be about 82% for the membrane both before and after surface modification. Surface modification does not appear to alter the fiber diameter distribution and porosity, and only slightly reduces the pore size and shifts the pore size distribution to the lower end. In addition, water contact angle was measured for the pristine electrospun RC membrane as well as for the functionalized membranes after grafting with PGMA and finally with MMAEX ligand as shown in FIG. 8B. The contact angle was measured to be <30° for the unmodified membrane indicating its hydrophilic nature. However, after ATRP grafting of PGMA, the membrane becomes moderately hydrophilic with contact angle at around 67°. After the final functionalization with MMAEX ligand, the membrane before moderately hydrophobic with a contact angle of abound 97°. This is likely due to the hydrophobic nature of phenylimidazole. The hydrophobicity of ligand plays an important role in the separation of full and empty capsids.

Zeta Potential Measurement of Full and Empty AAV2 Capsids. Zeta potential measurement was performed to determine the charges on the full and empty AAV2 capsids as shown in FIG. 9. Both empty and full AAV2 capsids have been purified following the clarification with BioOptimal, affinity capture with POROS CaptureSelect AAVX column, chromatographic separation using Applicant's MMAEX membrane, and finally buffer exchange. Based on the qPCR and ELISA measurements, the purity for both full and empty AAV2 are about 95% with the other 5% of the other capsid. Other impurities in the samples were not observed using SDS PAGE (not shown) except the bands for capsid proteins (VP1, VP2 and VP3). FIG. 9 shows that empty capsids are slightly positively charged compared to full capsids in agreement with previous studies. Moreover, the isoelectric points (pIs) for the full and empty capsids are at around 5.3 and 5.8 respectively with 0.5 pH unit difference in agreement with earlier results. Under the 20 mM BTP buffer at pH 9, the empty capsids remain slightly more positively charged.

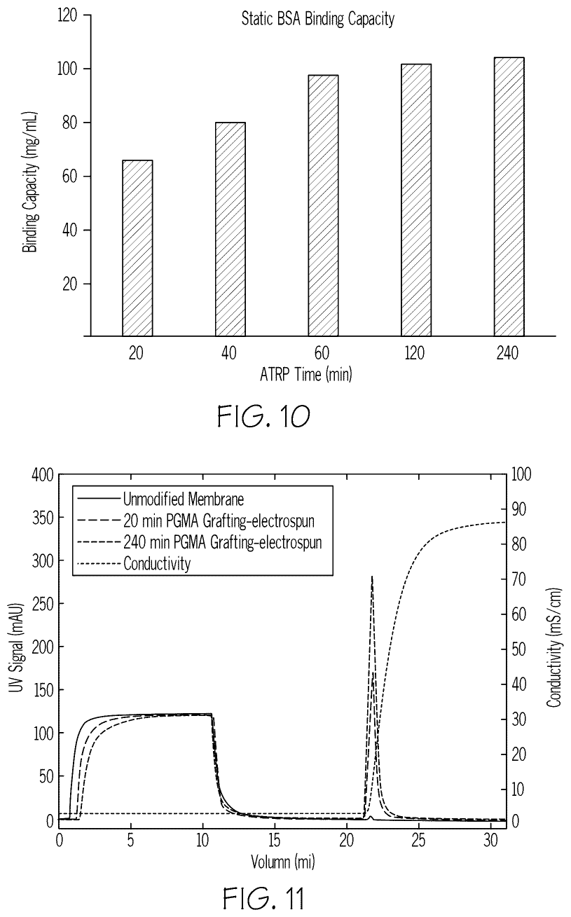

Protein Binding Capacities of Functionalized MMAEX Membrane. Before the functionalized MMAEX membrane was used for the AAV2 full and empty capsid separation, its static and dynamic binding capacities for bovine serum albumin (BSA) were determined. Since the MMAEX membrane is positively charged at neutral pH, negatively charged BSA was used as a model protein. For static binding, membranes with different ATRP grafting times were incubated with the samples containing 1 mg/mL BSA in 20 mM phosphate buffer at pH 7 for 7 hours. The amount of bound BSA were then determined by measuring the BSA concentrations before and after incubation. FIG. 10 shows the static BSA binding capacity of the membrane as a function of PGMA grafting time. As shown in FIG. 10, increasing grafting time leads to an increase in BSA binding capacity more pronounced for the first 60 min then more slowly from 60 to 240 min. This trend follows the grafting degree as shown in FIG. 3 to some extent. Longer chains have more buried binding sites that are likely inaccessible for protein binding. The static binding capacity reached close to 100 mg/mL after 60 min of ATRP reaction.

Dynamic binding capacity for BSA was also investigated for the MMAEX membrane. The experiments were performed for two ATRP grafting times at 20 and 240 mins using the FPLC bind and elute approach. The feed solutions contained 1 mg/mL BSA in 20 mM phosphate buffer with 0 mM NaCl at pH 7. A total of 10 mL of feed sample was pumped through the membranes at 1 mL/min flow rate following by the 10 mL of buffer wash. The elution buffer used 20 mM phosphate buffer with 1 M NaCl. The dynamic binding capacity was calculated based on the total amount of protein in mg bound on the membrane per mL of membrane volume. Protein recovery was determined based on the percentage of the amount of BSA eluted over the total amount of the BSA bound to the membrane per mL of membrane volume. FIG. 11 shows the FPLC chromatogram of the bind-and-elute of BSA samples using fabricated MMAEX membranes. The FPLC chromatogram for the unfunctionalized membrane was also shown. The dynamic binding capacities of the membranes at 20 and 240 min of ATRP time are shown in Table 2. It can be seen that dynamic binding capacity reached 67.6% for the MMAEX membrane with 20 min ATRP time. Increasing the ATRP time to 240 min increased the dynamic binding capacity marginally to only 77.5 mg/mL. This is probably due to the fact that only easily accessible binding sites are available for BSA binding during the FPLC runs. Recovery of the proteins are almost 100% for both membranes even though the surface of the membranes is moderately hydrophobic.

TABLE 2
The dynamic binding capacity and recovery of
BSA for the functionalized MMAEX membrane.

| | ATRP | Membrane | Dynamic Binding | |
|---|---|---|---|---|
| | Grafting | Volume | Capacity | Recovery |
| Substrate | Time (min) | (mL) | (mg/mL) | (%) |
| Electrospun | 20 | 0.012 | 67.6 | 96.3 |
| RC | 240 | 0.016 | 77.5 | 101.9 |

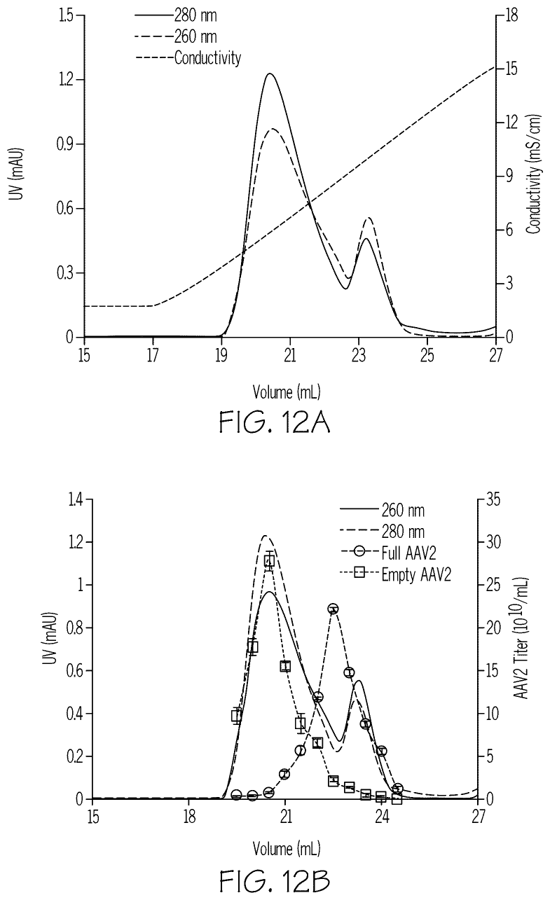

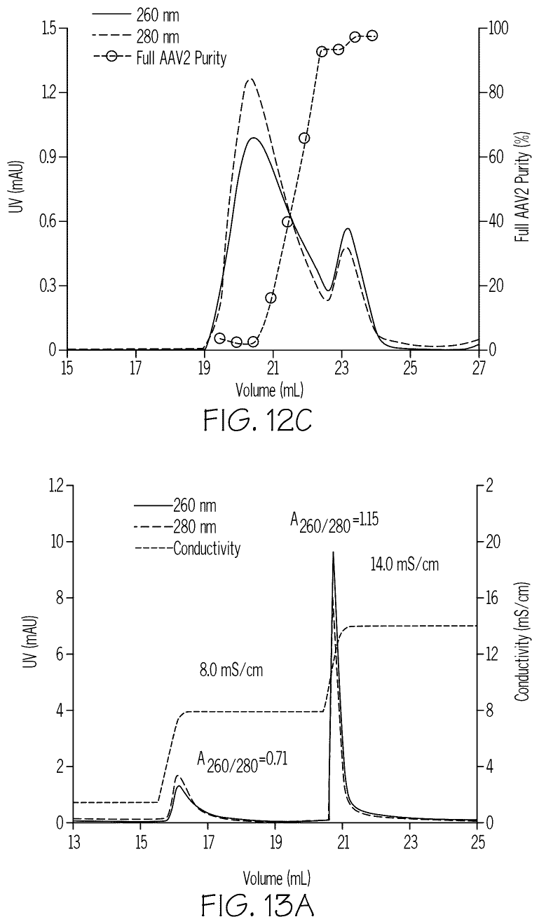

Separation of Full and Empty AAV2 Using Gradient Elution. After the functionalized MMAEX membrane was characterized, the separation of full and empty AAV2 was performed. In previous work, feed solutions containing synthetic feed mixtures of purified AAV2 full and empty capsids from the insect cell line Sf9 were used for the separation. Here actual AAV2 feed solutions after clarification of AAV2 lysate from mammalian HEK293 cell line and the subsequent affinity capture containing both full and empty capsids were used. The initial study used FPLC chromatography with gradient elution to separate the full and empty capsids similar to previous studies. The feed solution for the gradient elution has 1.389×10¹² total AAV2 capsids (measured by ELISA) in 10 mL of feed buffer consisting of 20 mM BTP buffer with 2 mM MgCl₂ at pH 9. The feed sample contained 0.440×10¹² full capsids based on qPCR. The total empty capsids were calculated to be 0.949×10¹² in the sample. The full capsid percentage in the feed was 31.7%. A total of 4 pieces of the functionalized membranes with total membrane volume of 0.025 mL was used for the gradient elution separation studies with a loading density of 5.556×10¹³ particles/mL of membrane volume, 60 times higher than the previous work with insect cell AAV2. The 10 mL feed solution was pumped through the membranes at a flow rate of 0.25 mL/min followed by 5 mL of buffer wash at the same flow rate. The elution was performed by varying the conductivity of the solution from the initial value of ˜1.5 mS/cm to the final value of ˜15.0 mS/cm with 10 mL of elution buffer as described above. FIG. 12A-12C shows UV 280 and 260 nm absorption peaks as well as the conductivity change (FIG. 12A), full and empty capsid titer (FIG. 12B) and full AAV2 purity in the eluted fractions (FIG. 12C).

The FPLC chromatogram in FIG. 12A shows the chromatogram during gradient elution where the conductivity increased from 1.7 to 15.0 mS/cm by mixing the binding or feed buffer (20 mM BTP with 2.0 mM MgCl₂ at pH 9) with elution buffer (20 mM BTP with 2.0 mM MgCl₂ at pH 9.0 with 1 M NaCl) from 0 linearly increased to 20%. It can be seen that two distinctive elution peaks appear in the chromatogram at corresponding conductivity of 6.3 and 10.1 mS/cm respectively for the first and second peak position. The measured A260/A280 ratios for the two peaks are 0.81 and 1.16 respectively. This suggests that the eluates from the first peak contain largely the empty capsids whereas the eluates from the second peak contain largely the full capsids. The titers of full and empty capsids for each fraction were quantified using qPCR and ELISA assays as shown in FIG. 12B. It can be seen that the first three fractions contain almost exclusively empty capsids. Starting from the fraction 4, full capsid concentration increases from 2.94×10¹⁰ vc/mL reaching a maximum value of 22.08×10¹⁰ vc/mL at fraction 7. Fractions 8-11 show a rapid decrease in full capsid titer to 1.21×10¹⁰ vc/mL at fraction 11. The empty capsid titer, on the other hand, is peaked at fraction 3 with 27.74×10¹⁰ vc/mL. Table 3 shows the full and empty capsid titers for the feed, flow through as well as eluate fraction 1-11. A total of 3.2% of the full capsids and 11.3% of the empty capsids were not bound to the membrane and ended up in the flow through fraction. The full capsid percentage reduced from 31.7% in the feed to 11.6% in the flow through. In the eluates, the AAV2 full capsid percentage increased from less than 5% in the first 3 fractions to 15.9, 39.0, 64.2, 90.6, 94.9, 95.2 and 94.5% in fractions 4-11 respectively as shown in FIG. 12C and listed in Table 3.

TABLE 3
Separation of full and empty AAV2 capsids under
gradient elution using MMAEX membrane.

| | | Full AAV2 | Empty AAV2 | Full AAV2 |
|---|---|---|---|---|
| | Volume | titer | titer | purity |
| | (mL) | (10¹⁰ vc/mL) | (10¹⁰ vc/mL) | (%) |
| Feed | 10.0 | 4.40 ± 0.24 | 9.49 ± 0.27 | 31.7 ± 0.8 |
| Flow Through | 10.0 | 0.14 ± 0.03 | 1.07 ± 0.14 | 11.6 ± 0.6 |
| Eluate Fraction | | | | |
| 1 | 0.5 | 0.34 ± 0.08 | 9.73 ± 0.91 | 3.4 ± 0.5 |
| 2 | 0.5 | 0.42 ± 0.07 | 17.74 ± 0.91 | 2.3 ± 0.2 |
| 3 | 0.5 | 0.72 ± 0.11 | 27.74 ± 1.24 | 2.5 ± 0.3 |
| 4 | 0.5 | 2.94 ± 0.37 | 15.58 ± 0.45 | 15.9 ± 1.3 |
| 5 | 0.5 | 5.67 ± 0.63 | 8.86 ± 1.11 | 39.0 ± 0.7 |
| 6 | 0.5 | 11.74 ± 0.18 | 6.55 ± 0.44 | 64.2 ± 1.2 |
| 7 | 0.5 | 22.08 ± 0.28 | 2.30 ± 0.21 | 90.6 ± 0.7 |
| 8 | 0.5 | 14.75 ± 0.28 | 1.43 ± 0.10 | 91.2 ± 0.4 |
| 9 | 0.5 | 8.83 ± 0.37 | 0.47 ± 0.12 | 94.9 ± 1.0 |
| 10 | 0.5 | 5.55 ± 0.33 | 0.28 ± 0.03 | 95.2 ± 0.2 |
| 11 | 0.5 | 1.21 ± 0.27 | 0.07 ± 0.01 | 94.5 ± 0.5 |

For the full capsids collected in the eluates, full capsid recovery and purity can be determined. Based on the eluate fraction 4-11 collected, a full capsid recovery of 82.7% at a purity of 67.2% can be obtained. Based on eluate fraction 5-11, a full capsid recovery of 79.4% at a purity of 77.8% can be obtained instead. The slight reduced recovery and purity obtained during gradient elution for HEK AAV2 compared to Applicant's previous Sf9 AAV2 of ˜93.6% full capsid recovery at 88.6% purity is partly due to the fact a loading density at 5.56×10¹³ vc/mL during current study is 60 times higher than the previous study at 0.93×10¹² vc/mL. Based on eluate fraction 6-11 collected, a full capsid recovery of 72.9% at 85.3% purity can be achieved. Further increase in purity can be obtained if only eluate fraction 7-11 were pooled reaching a full capsid recovery of 59.6% at 92.0% purity.

Separation of Full and Empty AAV2 using Two-step Elution. Two-step elution was also performed to separate the full and empty HEK AAV2 capsids after going through clarification of lysate and affinity capture unlike previous work with artificially mixed 1:1 ratio of Sf9 insect cell AAV2. Moreover, the loading density of the HEK AAV2 is 1.32×10¹⁴ vc/mL of membrane volume. Compared to the earlier loading density of 1.63×10¹³ vc/mL, it is more than 8 times higher than the previous study. For the two-step elution study, three first step elution conductivities at 8.0, 8.5 and 9.0 mS/cm were used, whereas the second step elution conductivity was kept constant at 14.0 mS/cm. The first-step conductivity values were chosen based partly on the gradient elution results, and partly on experimental trials.

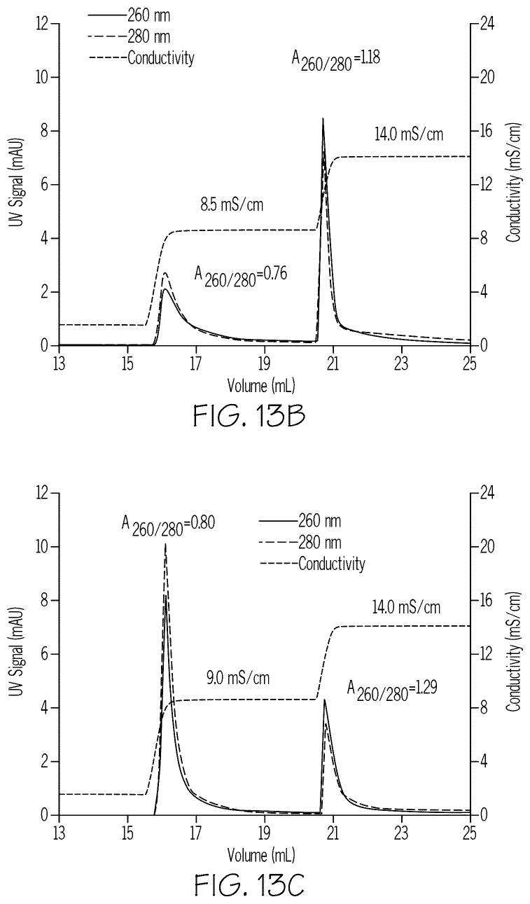

FIG. 13A shows the two-step elution chromatogram for separating the full and empty capsids with 1^{st} elution step conductivity at 8.0 mS/cm. Two elution peaks were observed corresponding to the absorbance peaks measured at 260 nm and 280 nm with the A260/A280 absorbance ratio of 0.71 and 1.15 for the 1^{st} and 2^{nd} elution steps respectively. Based on the absorbance ratio, it is clear that the eluates from the first elution peak is composed mainly of the empty capsids whereas the eluates from the second elution peak is made largely of full AAV capsids. When the 1^{st} elution step conductivity is increased to 8.5 and 9.0 mS/cm as shown in FIG. 13B and FIG. 13C respectively, the A260/A280 absorbance ratio for the first elution peak increases from 0.71 to 0.76 and 0.80 accordingly. This suggests that as the 1^{st} elution step conductivity increases, more full capsids are being eluted during the first step leading to a slight increase in the A260/A280 absorbance ratio. At the same time, the A260/A280 absorbance ratio for the second elution peak also increases from 1.15 to 1.18 and finally to 1.29 as the 1^{st} elution step conductivity increases from 8 to 8.5 and 9.0 mS/cm respectively. This indicates that more empty capsids are being eluted during the 1^{st} elution step and less empty capsids are being eluted during the 2nd elution step. The actual full capsid titers during the first and second elution steps, the full capsid recovery and full capsid purity for three different two-step elution runs are shown in Table 4.

TABLE 4
Full and total AAV2 capsid titers in each of the
5 mL eluate from the 2-step elution experiments.
Full AAV2 recovery and purity are also shown.

| | | Full AAV | Total AAV | | |
|---|---|---|---|---|---|
| Conductivity | | Capsid | Capsid | Full | Full |
| of 1^{st} Step | | Titer¹ | Titer² | AAV2 | AAV2 |
| Elution | Step | (×10¹⁰ | (×10¹⁰ | Recovery | Purity |
| (mS/cm) | Elution | vc/mL) | vc/mL) | (%) | (%) |
| 8.0 | 1^{st} | 1.4 ± 0.1 | 35.0 ± 0.8 | 6.4 ± 0.4 | 4.0 ± 0.3 |
| | 2^{nd} | 18.5 ± 0.6 | 31.3 ± 1.1 | 87.9 ± 2.9 | 59.2 ± 0.2 |
| 8.5 | 1^{st} | 3.0 ± 0.3 | 38.0 ± 2.1 | 14.1 ± 1.4 | 7.9 ± 0.4 |
| | 2^{nd} | 16.0 ± 0.9 | 20.5 ± 1.1 | 76.0 ± 2.0 | 77.9 ± 2.5 |
| 9.0 | 1^{st} | 4.8 ± 0.3 | 39.7 ± 1.5 | 22.9 ± 1.3 | 12.1 ± 0.2 |
| | 2^{nd} | 14.8 ± 1.0 | 16.1 ± 0.3 | 70.3 ± 2.5 | 92.2 ± 1.8 |

The results from Table 4 show that depending on the 1^{st} elution step conductivity, full AAV2 capsid recovery and purity from second step eluates can vary. When the 1^{st} elution step conductivity is 8.0 mS/cm, full AAV recovery at 87.9% at 59.2% purity can be achieved. A full AAV2 capsid recovery of 76.0% at 77.9% purity can be achieved when the 1^{st} elution step conductivity is increased to 8.5 mS/cm. Finally, when the 1^{st} step conductivity increases to 9.0 mS/cm, a full AAV capsid recovery at 70.3% at 92.2% purity can be achieved. Compared to gradient elution results with a full capsid recovery of 72.9% at 85.3% purity or a full capsid recovery of 59.6% at 92.0% purity, the two-step elution with 70.3% recovery at 92.2% purity appears to have a better performance, even though the loading density is twice as much. The performance of MMAEX for full and empty AAV2 separation with the two-step elution is also better with HEK AAV2, compared to previous full capsid recovery of 59.8% at 92.8% purity with Sf9 AAV2. Since the loading density is 8 times higher compared to Applicant's previous results, the full and empty capsid separation performance for HEK293 AAV2 is even more remarkable with the two-step elution process using Applicant's MMAEX membrane.

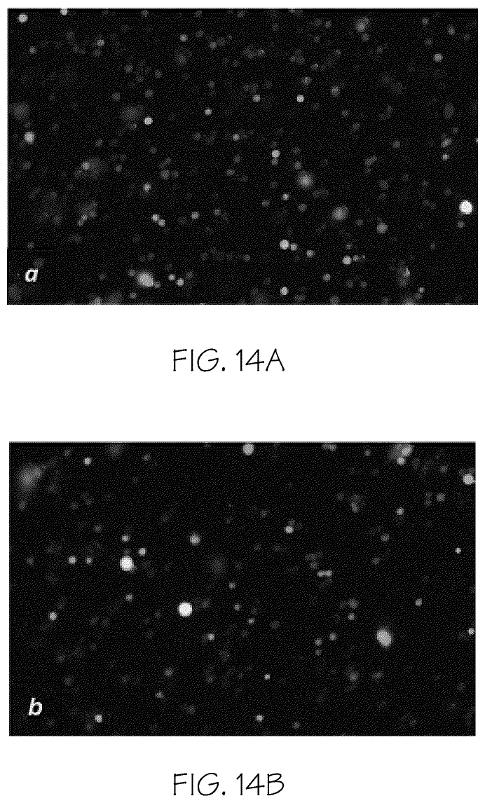

AAV Infectivity. In order to investigate the effects of the MMAEX ligand functionalized membrane on the infectivity of the AAV2 with enriched full capsids, infection of HEK293 cells by AAV2 both before and after chromatographic separation was investigated. The detailed procedure for the experiments were described earlier and the fluorescent images are shown in FIGS. 14A-14B. The total number of HEK cells, the full capsid AAV2 number measured by qPCR, and the infected HEK cell numbers are listed in Table 5. In addition, the infection percentage and the MOIs for both samples of AAV2 are also listed. It can be seen that the infection percentage of the HEK cells for the chromatographically purified AAV2 capsids is 27.9%, somewhat lower than the corresponding value of 39.9% before the MMAEX full and empty separation step. However, the initial full AAV2 capsid number is 0.84×10¹⁰ fewer than the corresponding number of 1.28×10¹⁰ before chromatographic separation. The overall MOIs for the two samples are almost the same within the error bar, 38.6×10³ vs 40.9×10³. MOI is a measure of the number of AAV particles needed in order to infect certain number of cells. It can be seen the MMAEX chromatographic separation does not affect the infectious quality of the AAV2 capsids.

TABLE 5
HEK infection by the AAV2 before and after MMAEX chromatographic
separation to remove the empty capsids.

| | Infected by AAV2 | Infected by AAV2 |
|---|---|---|
| | before MMAEX | after MMAEX |
| | Separation | Separation |
| Full AAV2 Capsids Used for | 1.28 | 0.84 |
| Infection (×10¹⁰) | | |
| Total HEK Cells (×10⁵) | 8.4 | 7.5 |
| Infected HEK Cells (×10⁵) | 3.35 ± 0.34 | 2.09 ± 0.28 |
| Infection Percentage (%) | 39.9 ± 4.1 | 27.9 ± 3.7 |
| MOI (×10³) | 38.6 ± 5.5 | 40.9 ± 7.8 |

Conclusions. A multi-modal 1-methyl-phenylimidazole AEX ligand has been successfully functionalized on electrospun RC membrane substrate. The fabricated MMAEX membrane is highly porous with an average pore size of about 0.5 μm. The functionalized membrane is slightly hydrophobic with a water contact angle of about ˜97°. The static binding capacity increases with the increase of grafting degree and can reach over ˜100 mg/mL for BSA. The dynamic binding capacity of BSA is at around ˜70 mg/mL for both high and low grafting degrees. Protein recovery is almost 100%.

These MMAEX membrane exhibit superior performance for the separation of full and empty AAV2 produced from HEK293 in-house. The clarified and affinity processed feedstock contains 31.7% full AAV2 capsids. A full capsid recovery of 79.4% at 77.8% purity can be obtained during gradient elution with a loading density of 5.56×10¹³ vc/mL, ˜60 times higher than a previous study using commercially purchased artificially mixed insect cell AA2 feed. A trade-off between purity and recovery is also observed. Alternatively, a full capsid recovery of 72.9% at 85.3% purity can also be obtained during gradient elution. In addition, two-step elution for the separation of full and empty capsids was also investigated. These results suggest that even better performance can be obtained with 70.3% recovery at 92.2% purity at a loading density of 1.32×10¹⁴ vc/mL when the first elution step conductivity is 9 mS/cm. This loading density is 2 times higher than that of the gradient elution, almost 8 times higher than the corresponding two-step elution studies with Sf9 AAV2 previously. The trade-off between recovery and purity is also observed when the first elution step conductivity is reduced to 8 mS/cm or 8.5 mS/cm where a 77.6% recovery at 79.0% purity or 87.9% recovery at 59.2% are obtained respectively. These results demonstrate superior performance for the full and empty AAV2 capsid separation after affinity capture.

Finally, infectivity studies of AAV2 capsids before and after the MMAEX chromatographic separation were performed. The MOIs for both samples are about the same at ˜40,000 indicating that MMAEX does not inactivate or damage infective full capsid AAV2. These results indicate the superior performance of this MMAEX membrane and its potential industrial applications.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those of ordinary skill in the art may better understand the aspects of the disclosure. Those of ordinary skill in the art should appreciate that they may readily use the disclosure as a basis for designing or modifying other processes and structures for carrying out the same purposes and/or achieving the same advantages of the embodiments introduced herein. Those of ordinary skill in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim is an open group. The terms “a”, “an”, and other singular terms are intended to include the plural forms thereof unless specifically excluded.

Conditional language used herein, such as, among others, “can”, “might”, “may”, “e.g.”, and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements, and/or states are in any way required for one or more embodiments.

While the above detailed description has shown, described, and pointed out novel features as applied to various embodiments, it will be understood that various omissions, substitutions, and changes in the form and details of the embodiments illustrated can be made without departing from the spirit of the disclosure. As will be recognized, the various embodiments described herein can be embodied within a form that does not provide all the features and benefits set forth herein, as some features can be used or practiced separately from others. The scope of protection is defined by the appended claims rather than by the foregoing description. All changes which come within the meaning and range of equivalency of the claims are to be embraced within their scope.

What is claimed is:

1. A method of separating materials, the method comprising:
associating the materials with a surface comprising a ligand, the ligand comprising:
a hydrophobic unit; and
an anionic unit;
forming a material-ligand complex;
washing the surface; and
eluting the materials from the material-ligand complex.

2. The method of claim 1, wherein the associating comprises incubating the materials with the surface.

3. The method of claim 1, wherein the associating comprises flowing the materials through the surface.

4. The method of claim 1, wherein the materials are selected from the group consisting of nucleic acids, DNA, RNA, mRNA, miRNA, monoclonal antibodies, vaccines, therapeutic proteins, viruses, viral particle capsids, or combinations thereof.

5. The method of claim 1, wherein the materials comprise viral particle capsids.

6. The method of claim 5, wherein the viral particle capsids are selected from the group consisting of full capsids, empty capsids, adeno-associated virus (AAV) capsids serotypes, AAV2 capsids, AAV5 capsids, AAV9 capsids, or combinations thereof.

7. The method of claim 1, wherein the surface is selected from the group consisting of beads, resin beads, columns, membranes, regenerated cellulose (RC) membranes, electrospun nanofiber RC membranes, chromatographic stationary phases, monolith surfaces, organic substrates, inorganic substrates, or combinations thereof.

8. The method of claim 1, wherein the hydrophobic unit is selected from the group consisting of aromatic groups, phenyl groups, aromatic conjugated groups, aliphatic groups, or combinations thereof.

9. The method of claim 1, wherein the anionic unit comprises quaternary amine groups.

10. The method of claim 1, wherein the anionic unit comprises imidazole groups.

11. The method of claim 1, wherein the ligand comprises a cationic charge.

12. The method of claim 1, wherein the ligand comprises phenylimidazole cations.

13. A surface operational to separate materials, the surface comprising a ligand operable to associate with the materials, and wherein the ligand comprises a hydrophobic unit and an anionic unit.

14. The surface of claim 13, wherein the ligand is operable to bind to materials selected from the group consisting of nucleic acids, DNA, RNA, mRNA, miRNA, monoclonal antibodies, vaccines, therapeutic proteins, viruses, viral particle capsids, or combinations thereof.

15. The surface of claim 13, wherein the materials comprise viral particle capsids.

16. The surface of claim 15, wherein the viral particle capsids are selected from the group consisting of full capsids, empty capsids, adeno-associated virus (AAV) capsids serotypes, AAV2 capsids, AAV5 capsids, AAV9 capsids, or combinations thereof.

17. The surface of claim 13, wherein the surface is selected from the group consisting of beads, resin beads, columns, membranes, regenerated cellulose (RC) membranes, electrospun nanofiber RC membranes, chromatographic stationary phases, monolith surfaces, organic substrates, inorganic substrates, or combinations thereof.

18. The surface of claim 13, wherein the hydrophobic unit is selected from the group consisting of aromatic groups, phenyl groups, aromatic conjugated groups, aliphatic groups, or combinations thereof.

19. The surface of claim 13, wherein the anionic unit comprises at least one of a quaternary amine group or an imidazole group.

20. The surface of claim 13, wherein the ligand comprises at least one of a cationic charge or a phenylimidazole cation.
